(19) United States
(12) Patent Application Publication
(10) Pub. No.: US 2013/0071341 A1
(43) Pub. Date: Mar. 21, 2013

(54) SUNSCREEN COSMETIC

(71) Applicant: Kahori Ishida, Yokohama-Shi (JP)

(72) Inventor: Kahori Ishida, Yokohama-Shi (JP)

(21) Appl. No.: 13/635,724

(22) PCT Filed: Mar. 24, 2010
(86) PCT No.: PCT/JP2010/055028

§ 371 Date: Oct. 24, 2012

(51) Int. Cl. A61K 8/37; A61Q 17/04

(52) U.S. Cl. 424/60

(57) ABSTRACT

The present invention aims to provide a sunscreen cosmetic that does not contain diethylamino hydroxybenzoyl hexyl benzoate, can absorb a wide range of wavelengths of UV rays across the UVA and UVB regions, and prevent staining (dyeing) due to secondary adhesion to clothing. The present invention relates to a sunscreen cosmetic containing 2-hydroxy-4-methoxybenzophenone, 2-ethylhexyl p-methoxycinnamate, and octocrylene, a volatile hydrocarbon oil, and zinc oxide and/or titanium oxide hydrophobized with an agent other than fatty acids.

### TECHNICAL FIELD

The present invention relates to a sunscreen cosmetic. More particularly, the present invention relates to a sunscreen cosmetic that reduces staining (dyeing) due to secondary adhesion to clothing.

### BACKGROUND ART

The important wavelength regions of UV rays absorbed by a sunscreen cosmetic are UV-A region (320-400 nm) and UV-B region (290-320 nm). It has been believed that the UV ray in the UV-A region browns skin, but does not cause sunburn and promote aging of skin, unlike the UV ray in the UV-B region.

However, in recent years, it has been found that in contrast to the UV ray in the UV-B region that remains in a relatively superficial area of skin, the UV ray in the UV-A region reaches a deep area of skin, and may become a reason for triggering not only aging of skin but also skin carcinoma. Thus, for sunscreen cosmetics, there is an increasing need for absorbing the UV ray especially in the UV-A region.

Diethylamino hydroxybenzoyl hexyl benzoate is known as one of the UV absorbing agents effective for absorbing the UV ray in the UV-A region. However, diethylamino hydroxybenzoyl hexyl benzoate has a problem that it leads to staining of clothing due to secondary adhesion.

To solve the above problem, Patent Document 1 discloses the technique to seek to prevent staining by combining diethylamino hydroxybenzoyl hexyl benzoate with certain benzotriazole derivatives to form a sunscreen cosmetic.

In addition, Patent Document 2 describes that secondary adhesion (staining) to clothing can be prevented in a sunscreen cosmetic containing diethylamino hydroxybenzoyl hexyl benzoate, by compounding certain ester oils and zinc oxide and/or titanium oxide hydrophobized with an agent other than fatty acids to form a water-in-oil emulsion composition.

Although efforts to reduce staining (dyeing) property of sunscreen cosmetics are thus carried out by, for example, combining a sunscreen cosmetic containing diethylamino hydroxybenzoyl hexyl benzoate with various other ingredients, problems have arisen that diethylamino hydroxybenzoyl hexyl benzoate (for example, Uvinul A plus (product name)) can not be compounded in cosmetics sold in U.S., whereas without compounding diethylamino hydroxybenzoyl hexyl benzoate, absorbing of the UV ray in the UVA region is not sufficient, leading to low PFA.

### PRIOR ART DOCUMENTS

### Patent Documents

Patent Document 1: JP-A 2007-182388
Patent Document 2: JP-A 2009-206473

### SUMMARY OF THE INVENTION

### Problem to be Solved by the Invention

Therefore, the problem in the present invention is to provide a sunscreen cosmetic that does not contain diethylamino hydroxybenzoyl hexyl benzoate, can absorb a wide range of wavelengths of UV rays across the UVA and UVB regions, and prevent staining (dyeing) due to secondary adhesion to clothing.

### Means for Solving the Problem

The present inventors, as a result of diligent research aimed at solving the above mentioned problem, have found that a sunscreen cosmetic containing a certain combination of UV absorbing agents, a volatile hydrocarbon oil, and an UV scattering agent achieves high SPF and PFA, can prevent staining (dyeing) to clothing, and has excellent comfortability, and thus have accomplished the present invention.

In other words, the present invention provides a sunscreen cosmetic comprising 2-hydroxy-4-methoxybenzophenone, 2-ethylhexyl p-methoxycinnamate and octocrylene, and a volatile hydrocarbon oil, and zinc oxide and/or titanium oxide hydrophobized with an agent other than fatty acids.

### Effects of the Invention

The sunscreen cosmetic of the present invention can successfully absorb a wide range of wavelengths of UV rays across the UVA and UVB regions, and does not cause a problem of secondary adhesion (staining) to clothing. Furthermore, a volatile hydrocarbon oil contained in the sunscreen cosmetic of the present invention provides a soft feeling when using.

### BRIEF DESCRIPTION OF THE DRAWING

FIG. 1 is an illustration of a method for measuring staining in Examples.

### MODES FOR CARRYING OUT THE INVENTION

UV absorbing agents contained as essential ingredients in the sunscreen cosmetic of the present invention are a combination of three substances; 2-hydroxy-4-methoxybenzophenone, 2-ethylhexyl p-methoxycinnamate and octocrylene.

2-Hydroxy-4-methoxybenzophenone (labeling name: oxybenzone-3) is an UV absorbing agent having a wide absorption band across the UVA and UVB regions.

The content of 2-hydroxy-4-methoxybenzophenone in the cosmetic of the present invention is preferably 0.3-5% by mass, more preferably 0.5-3% by mass, and further preferably 1-3% by mass.

The substance 2-ethylhexyl p-methoxycinnamate (octyl methoxycinnamate) is an UV absorbing agent based on methoxycinnamate, having an absorption band in the UVB region.

The content of 2-ethylhexyl p-methoxycinnamate in the cosmetic of the present invention is preferably 0.5-15% by mass, more preferably 1-10% by mass, and further preferably 3-8% by mass.

Octocrylene is a diphenyl compound having an absorption band in the UVB region, which is in particular an UV absorbing agent with superior photostability.

The content of octocrylene in the cosmetic of the present invention is preferably 0.5-15% by mass, more preferably 1-10% by mass, and further preferably 3-8% by mass.

The sunscreen cosmetic of the present invention contains a volatile hydrocarbon oil as an essential ingredient, in addition to the above UV absorbing agents.

Volatile hydrocarbon oils used in the present invention are not limited especially, and one or more oils selected from those typically used for such as cosmetics as appropriate may be used. For example, they are hydrocarbon oils that volatilize at room temperature (25° C.) and normal pressure (1 atm: 9.8×10⁴ Pa), and specifically, they include, for example, isodecane, isododecane, isohexadecane and combinations thereof.

The content of a volatile hydrocarbon oil in the cosmetic of the present invention is preferably 0.5-50% by mass, more preferably 3-30% by mass, and further preferably 5-20% by mass.

The sunscreen cosmetic of the present invention contains, as an essential ingredient, zinc oxide and/or titanium oxide hydrophobized with an agent other than fatty acids, in addition to the above UV absorbing agents and the volatile hydrocarbon oils.

Zinc oxide and titanium oxide are inorganic ingredients that are conventionally contained in cosmetics and the like, but preferably used in the present invention are those hydrophobized on their surfaces with hydrophobizing agents other than fatty acids. Zinc oxide and titanium oxide hydrophobized with fatty acids, in particular those hydrophobized with stearic acids or myristic acids are excluded from the present invention. Furthermore, in the present invention, zinc oxide and titanium oxide not hydrophobized on their surfaces are also included in “zinc oxide and/or titanium oxide hydrophobized with agents other than fatty acids”.

As zinc oxide and titanium oxide, those having a particle size where the short side length is 8-30 nm and the long side length is 50-100 nm, are particularly preferred.

Hydrophobizing agents other than fatty acids include higher alcohol, hydrocarbon, triglyceride, ester, silicone oil, silicone resin, fluorine compounds. Specific examples include alkyl triethoxysilane, alkyl trimethoxysilane, perfluoroalkyl phosphate, (alkyl acrylate/dimethyl silicone) copolymer, dextrin palmitate, triethoxysilylethylpolydimethylsiloxyethylhexyldimethicone, hydrogen dimethicone, dimethicone, polymeric silicone, sodium acryloyl dimethyl taurate/methacrylamide lauric acid copolymer and the like.

The content of zinc oxide and/or titanium oxide hydrophobized with agents other than fatty acids in the cosmetic of the present invention is preferably 1-30% by mass, more preferably 3-20% by mass, and further preferably 5-15% by mass.

The sunscreen cosmetic of the present invention may contain other ingredients without impairing the effect of the present invention, in addition to the above essential ingredients.

Specific examples of ingredients that can be contained in the sunscreen cosmetic will be shown below, but are not limited to them.

Oil ingredients (except a volatile hydrocarbon oil) such as decamethyl cyclopentasiloxane, isononyl isononanoate, dimethyl polysiloxane, heptamethyl octyl trisiloxane, trimethylsiloxysilicate, liquid paraffin, squalene, avocado oil, macadamia nut oil, corn oil, olive oil, canola oil, evening primrose oil, castor oil, sunflower seed oil, tea oil, rice bran oil, jojoba oil, cacao butter, palm oil, squalene, beef tallow, japan wax, bees wax, candelilla wax, carnauba wax, spermaceti, lanolin, liquid paraffin, polyoxyethylene (8 mol) oleyl alcohol ether, glyceryl monooleate, cyclomethicone, dimethyl polysiloxane, and diphenyl polysiloxane.

Higher alcohols such as capryl alcohol, lauryl alcohol, myristyl alcohol, cetyl alcohol, cholesterol, and phytosterol.

Higher fatty acids such as capric acid, lauric acid, myristic acid, palmitic acid, stearic acid, behenic acid, lanolin fatty acid, linoleic acid, and linolenic acid.

Moisturizing agents such as polyethylene glycol, glycerin, sorbitol, xylitol, maltitol, mucopolysaccharide, hyaluronic acid, chondroitin sulfuric acid, and chitosan.

Thickening agents such as methyl cellulose, ethyl cellulose, gum arabic, and polyvinyl alcohol.

Liquid media constituting aqueous phase, such as ethanol, and 1,3-butylene glycol.

Antioxidants such as butyl hydroxytoluene, tocopherol, and phytic acid.

Antibacterial preservatives such as benzoic acid, salicylic acid, sorbic acid, paraoxybenzoic acid ester (such as ethylparaben, butylparaben), and hexachlorophene.

Amino acids such as glycine, alanine, valine, leucine, serine, threonine, phenyl alanine, tyrosine, aspartic acid, asparagine, glutamine, taurine, arginine, and histidine and salts thereof.

Organic acids such as acylsarcosine acid (for example, sodium lauroyl sarcosine), glutathione, citric acid, malic acid, tartaric acid, and lactic acid.

Vitamins such as Vitamin A and its derivatives, B vitamins such as vitamin B6 hydrochloride, vitamin B6 tripalmitate, vitamin B6 dioctanoate, vitamin B2 and its derivatives, vitamin B12, and vitamin B15 and its derivatives, C vitamins such as ascorbic acid, ascorbic acid phosphate ester (salt), and ascorbic acid dipalmitate, E vitamins such as α-tocopherol, β-tocopherol, γ-tocopherol, vitamin E acetate, and vitamin E nicotinate, D vitamins, vitamin H, pantothenic acid, pantethine.

Various kinds of agents such as nicotinic acid amide, benzyl nicotiniate, γ-orizanol, allantoin, glycyrrhizic acid (salt), glycyrrhetinic acid and its derivatives, hinokitiol, musizin, bisabolol, eucalyptol, thymol, inositol, saponins (such as saikosaponin, carrot saponin, luffa saponin, soapberry saponin), pantothenyl ethyl ether, ethynyl estradiol, tranexamic acid, cepharanthine, and placenta extract.

Natural extracts extracted with organic solvent, alcohol, polyalcohol, water, aqueous alcohol and the like, such as sorrel, Sophora flavescens, nuphar, orange, sage, thyme, yarrow, mallow, Cnidium officinale Makino, Swertia japonica Makino, Angelica sinensis, Picea jezoensis var. hondoensis, birch, Equisetum arvense, luffa, horse chestnut, Saxifraga stolonifera, Arnica montana, lily, mugwort, peony, aloe, gardenia, and Sawara cypress.

Cationic surfactants such as stearyl trimethyl ammonium chloride, benzalkonium chloride, and lauryl amine oxide.

Sequestering agents such as disodium edetate, trisodium edetate, sodium citrate, sodium polyphosphate, sodium metaphosphate, and gluconic acid.

The sunscreen cosmetic of the present invention can be provided in the form of, for example without limitation, an ointment, a cream, an emulsion, a lotion. Cosmetics in each form can be manufactured by usual method known in the art.

### EXAMPLES

Next, the present invention will be described more specifically with Examples, but the present invention is not limited to these Examples. It is noted that the content means % by mass relative to the total amount, unless otherwise indicated.

The water-in-oil sunscreen emulsion cosmetics (sunscreens in the form of emulsion) with compositions shown in tables 1 and 2 below were manufactured by a routine method. For cosmetics in each example, the washing/removing effect against staining was measured by the following method.

“Method for measuring staining (dyeing property)”

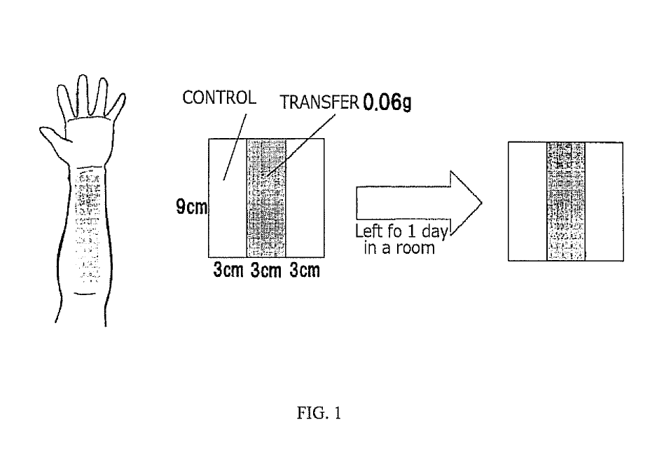

As shown in FIG. 1, a sample was applied thickly to the arm, and transferred to the middle of a piece of cotton broadcloth (the transferred amount was about 0.06 g). After leaving it for 1 day in a room, the piece of cloth was washed using a customary detergent for clothing (*1), the washing performance was determined visually.

*1: Attack (Kao Corporation)

### <Criteria for Determination>

◯: Staining was washed out to the extent that it can not be recognized visually.

×: Staining was observed even after washing.

A sensory test by professional panelists (10) for cosmetic products was also carried out to evaluate the feeling (moistness) when cosmetics in each example were applied to the skin, according to the following criteria.

### <Criteria for Evaluation>

◯: Determined as moist by more than or equal to 5 of 10 panelists.

×: Determined as moist by less than 5 of 10 panelists.

SPF and PFA for cosmetics in each example were also measured following the standard method. The results of the above measurement (evaluation) are shown in combination in tables 1 and 2.

TABLE 1

| | Comparative | Comparative | Comparative | Example | Example |
|---|---|---|---|---|---|
| | example 1 | example 2 | example 3 | 1 | 2 |
| Decamethyl cyclopentasiloxane | 15 | 0 | 0 | 0 | 0 |
| Isododecane | 0 | 15 | 15 | 15 | 15 |
| 2-hydroxy-4-methoxybenzophenone | 2 | 2 | 2 | 2 | 2 |
| 2-ethylhexyl p-methoxycinnamate | 5 | 5 | 5 | 5 | 5 |
| Octocrylene | 5 | 5 | 5 | 5 | 5 |
| Dimethyl polysiloxane | 5 | 5 | 5 | 5 | 5 |
| Glycerin | 5 | 5 | 5 | 5 | 5 |
| Dipropylene glycol | 5 | 5 | 5 | 5 | 5 |
| Lauryl PEG-9 | 3 | 3 | 3 | 3 | 3 |
| polydimethylsiloxyethyl dimethicone | | | | | |
| Ion exchange water | q.s. to 100 | | | | |
| Dimethyldistearyl ammonium hectorite | 2 | 2 | 2 | 2 | 2 |
| Trisodium edetate | 0.05 | 0.05 | 0.05 | 0.05 | 0.05 |
| Phenoxyethanol | 0.35 | 0.35 | 0.35 | 0.35 | 0.35 |
| Myristic acid-treated zinc oxide | 10 | 10 | — | — | — |
| Stearic acid-treated titanium oxide | — | — | 10 | — | — |
| Dextrin palmitate-treated zinc oxide | — | — | — | 10 | — |
| Alkyl triethoxysilane-treated zinc oxide | — | — | — | — | 10 |
| Alkyl triethoxysilane-treated titanium oxide | — | — | — | — | — |
| Fluorine-treated zinc oxide | — | — | — | — | — |
| Sodium acryloyl dimethyl | — | — | — | — | — |
| taurate/methacrylamide lauric acid | | | | | |
| copolymer-treated titanium oxide | | | | | |
| Polymeric silicone-treated zinc oxide | — | — | — | — | — |
| Hydrogen dimethicone-treated zinc oxide | — | — | — | — | — |
| | | | | — | — |
| Determination (visual) | x | x | x | ∘ | ∘ |
| Feeling of use (moistness) | x | ∘ | ∘ | ∘ | ∘ |
| SPF | 54 | 54 | 54 | 54 | 54 |
| PFA | 10 | 10 | 10 | 10 | 10 |

TABLE 2

| | Example | Example | Example | Example | Example |
|---|---|---|---|---|---|
| | 3 | 4 | 5 | 6 | 7 |
| Decamethyl cyclopentasiloxane | 0 | 0 | 0 | 0 | 0 |
| Isododecane | 15 | 15 | 15 | 15 | 15 |
| 2-hydroxy-4-methoxybenzophenone | 2 | 2 | 2 | 2 | 2 |
| 2-ethylhexyl p-methoxycinnamate | 5 | 5 | 5 | 5 | 5 |
| Octocrylene | 5 | 5 | 5 | 5 | 5 |
| Dimethyl polysiloxane | 5 | 5 | 5 | 5 | 5 |
| Glycerin | 5 | 5 | 5 | 5 | 5 |
| Dipropylene glycol | 5 | 5 | 5 | 5 | 5 |
| Lauryl PEG-9 | 3 | 3 | 3 | 3 | 3 |
| polydimethylsiloxyethyl dimethicone | | | | | |
| Ion exchange water | q.s. to 100 | | | | |
| Dimethyldistearyl ammonium hectorite | 2 | 2 | 2 | 2 | 2 |
| Trisodium edetate | 0.05 | 0.05 | 0.05 | 0.05 | 0.05 |
| Phenoxyethanol | 0.35 | 0.35 | 0.35 | 0.35 | 0.35 |
| Myristic acid-treated zinc oxide | — | — | — | — | — |
| Stearic acid-treated titanium oxide | — | — | — | — | — |
| Dextrin palmitate-treated zinc oxide | — | — | — | — | — |
| Alkyl triethoxysilane-treated zinc oxide | — | — | — | — | — |
| Alkyl triethoxysilane-treated titanium oxide | 10 | — | — | — | — |
| Fluorine-treated zinc oxide | — | 10 | — | — | — |
| Sodium acryloyl dimethyl | — | — | 10 | — | — |
| taurate/methacrylamide lauric acid | | | | | |
| copolymer-treated titanium oxide | | | | | |
| Polymeric silicone-treated zinc Oxide | — | — | — | 10 | |
| Hydrogen dimethicone-treated zinc oxide | — | — | — | — | 10 |
| Determination (visual) | ∘ | ∘ | ∘ | ∘ | ∘ |
| Feeling of use (moistness) | ∘ | ∘ | ∘ | ∘ | ∘ |
| SPF | 54 | 54 | 54 | 54 | 54 |
| PFA | 10 | 10 | 10 | 10 | 10 |

The cosmetics in Examples 1-7 and Comparative examples 1-3 containing the UV absorbing agents in combination of 2-hydroxy-4-methoxybenzophenone, 2-ethylhexyl p-methoxycinnamate and octocrylene, and zinc oxide and/or titanium oxide hydrophobized with various kinds of agents showed high SPF and PFA.

However, Comparative example 1 not containing a volatile hydrocarbon oil (isododecane) lacked moistness, and in Comparative examples 1-3 containing zinc oxide or titanium oxide hydrophobized with fatty acids, the preventive effect against staining was not observed.

Formulation examples for the sunscreen cosmetic of the present invention are shown below. All were the sunscreen cosmetics that had the preventive effect against staining and exerted a superior UV ray-absorbing ability.

### Example 8

### W/O Type Sunscreen Cosmetic (Sunscreen)

| | (% by mass) |
|---|---|
| isododecane | 18 |
| dimethyl polysiloxane | 2 |
| lauryl PEG-9 polydimethylsiloxyethyl dimethicone | 1.5 |
| trimethylsiloxysilicate | 5 |
| diisopropyl sebacate | 15 |
| 2-ethylhexyl p-methoxycinnamate | 7.5 |
| octocrylene | 0.5 |
| 2-hydroxy-4-methoxybenzophenone | 2 |
| dimethyldistearyl ammonium hectorite | 0.5 |
| dextrin palmitate-treated zinc oxide | 1 |
| purified water | balance |
| trisodium edetate | 0.05 |
| dipropylene glycol | 5 |
| paraben | 0.25 |
| glycerin | 2 |
| ethanol | 2 |
| phenoxyethanol | 0.3 |

### Example 9

### W/O Type Sunscreen Cosmetic (Sunscreen)

| | (% by mass) |
|---|---|
| isohexadecane | 25 |
| dimethyl polysiloxane | 5 |
| PEG-10dimethicone | 3 |
| trimethylsiloxysilicate | 5 |
| tripropylene glycol dineopentanoate | 15 |
| 2-ethylhexyl p-methoxycinnamate | 7.5 |
| octocrylene | 3 |
| 2-hydroxy-4-methoxybenzophenone | 1 |
| dimethyldistearyl ammonium hectorite | 0.2 |
| alkyl triethoxysilane-treated zinc oxide | 15 |
| purified water | balance |
| trisodium edetate | 0.05 |
| dipropylene glycol | 5 |
| paraben | 0.25 |
| glycerin | 2 |
| ethanol | 2 |
| phenoxyethanol | 0.3 |

### Example 10

### WO Type Sunscreen Cosmetic (Sunscreen)

| | (% by mass) |
|---|---|
| isododecane | 5 |
| dimethyl polysiloxane | 2 |
| PEG-9 polydimethylsiloxyethyl dimethicone | 3.5 |
| trimethylsiloxysilicate | 5 |
| glyceryl tri (2-ethylhexanoate) | 1.5 |
| 2-ethylhexyl p-methoxycinnamate | 5 |
| octocrylene | 5 |
| 2-hydroxy-4-methoxybenzophenone | 0.3 |
| dimethyldistearyl ammonium hectorite | 2.5 |
| fluorine-treated zinc oxide | 10 |
| purified water | balance |
| trisodium edetate | 0.5 |
| dipropylene glycol | 5 |
| paraben | 0.25 |
| glycerin | 2 |
| ethanol | 2 |
| phenoxyethanol | 0.3 |

### Example 11

### W/O Type Sunscreen Cosmetic (Sunscreen)

| | (% by mass) |
|---|---|
| isohexadecane | 7 |
| dimethyl polysiloxane | 2 |
| sorbitan sesquiisostearate | 1.5 |
| trimethylsiloxysilicate | 5 |
| pentaerythrityl tetra2-ethylhexanoate | 10 |
| 2-ethylhexyl p-methoxycinnamate | 0.5 |
| octocrylene | 15 |
| 2-hydroxy-4-methoxybenzophenone | 1.5 |
| dimethyldistearyl ammonium hectorite | 2 |
| dicaprylyl carbonate | 10 |
| polymeric silicone-treated zinc oxide | 30 |
| purified water | balance |
| trisodium edetate | 0.05 |
| dipropylene glycol | 5 |
| paraben | 0.25 |
| glycerin | 2 |
| ethanol | 2 |
| phenoxyethanol | 0.3 |

### Example 12

### W/O Type Sunscreen Cosmetic (Sunscreen)

| | (% by mass) |
|---|---|
| isododecane | 40 |
| polyglyceryl-2 diisostearate | 3 |
| 2-ethylhexyl p-methoxycinnamate | 15 |
| octocrylene | 10 |
| 2-hydroxy-4-methoxybenzophenone | 5 |
| pentaerythrityl tetra2-ethylhexanoate | 5 |
| diethyl aminohydroxybenzoyl hexyl benzoate | 5 |
| diisopropyl sebacate | 10 |
| alkyl triethoxysilane-treated titanium oxide | 15 |
| purified water | balance |
| trisodium edetate | 0.1 |
| dipropylene glycol | 5 |
| paraben | 0.3 |
| glycerin | 10 |
| ethanol | 3 |
| phenoxyethanol | 0.35 |
| isopropyl palmitate | 15 |

1. A sunscreen cosmetic composition, comprising:
a component of 2-hydroxy-4-methoxybenzophenone;
a component of 2-ethylhexyl p-methoxycinnamate; and
a component of octocrylene;
a component of a volatile hydrocarbon oil; and
a component of zinc oxide and/or titanium oxide hydrophobized with a non-fatty-acid hydrophobic agent.

2. The sunscreen cosmetic composition, according to claim 1, wherein:
the content of 2-hydroxy-4-methoxybenzophenone is 0.3-5% by mass;
the content of 2-ethylhexyl p-methoxycinnamate is 0.5-15% by mass;
the content of octocrylene is 0.5-15% by mass;
the content of the volatile hydrocarbon oil is 0.5-50% by mass; and
the content of the zinc oxide and/or titanium oxide hydrophobized with a non-fatty-acid hydrophobic agent, is 1-30% by mass.
